(19) United States
(12) Patent Application Publication
(10) Pub. No.: US 2026/0049986 A1
(43) Pub. Date: Feb. 19, 2026

(54) Antigens for Detection of Early Lyme Disease

(71) Applicants: Antigen Discovery Inc., Irvine, CA (US); Academisch Medisch Centrum, Amsterdam (NL)

(72) Inventors: Joppe Willem Robert Hovius, Amsterdam (NL); Abhijeet Nayak, Amsterdam (NL); Arlo Randall, Huntington Beach, CA (US); Gary Hermanson, Encinitas, CA (US); Angela Yee, Irvine, CA (US)

(73) Assignees: Antigen Discovery Inc., Irvine, CA (US); Academisch Medisch Centrum, Amsterdam (NL)

(21) Appl. No.: 18/854,014

(22) PCT Filed: Apr. 3, 2023
(86) PCT No.: PCT/US2023/065283

§ 371 Date: Oct. 3, 2024

Related U.S. Application Data
(60) Provisional application No. 63/327,234, filed on Apr. 4, 2022.

(51) Int. Cl. G01N 33/569; A61K 39/00; A61K 39/02; C07K 14/20

(52) U.S. Cl. CPC G01N 33/56911; A61K 39/0225; C07K 14/20; A61K 2039/53; G01N 2333/20; G01N 2469/20

(57) ABSTRACT

Selected Antigens for early detection of Lyme disease are presented, and particularly for Lyme disease caused by B. afzelii. Advantageously, these antigens allow for unprecedented and unexpectedly high sensitivity and specificity in detection of early Lyme disease.

This application claims priority to our U.S. provisional patent application with the Ser. No. 63/327,234, which was filed Apr. 4, 2022, and which is incorporated by reference herein.

### SEQUENCE LISTING

The content of the XML file of the sequence listing named 101519.0010PCT.xml, which is 11 KB in size, was created on Mar. 24, 2023 and electronically submitted via Patent Center along with the present application and is incorporated by reference in its entirety.

### FIELD OF THE INVENTION

The field of the invention is diagnostic compositions and methods, especially as it relates to detection of early Lyme disease.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Lyme disease, the most prevalent and debilitating vector-borne zoonotic disease in the Northern hemisphere, is caused by a group of spirochete bacteria belonging to Borrelia burgdorferi sensu lato genospecies (amongst which B. burgdorferi, B. afzelii and B. garinii), with B. burgdorferi being the dominant species in the United States and B. afzelii in Europe. These Borrelia genospecies are transmitted by hard bodied Ixodes ticks in a complex enzootic cycle involving small mammals and birds. When humans contract an infection, it can result in a multi-system disorder manifested by diverse clinical signs. In the skin, symptoms include erythema migrans (“EM”), a bull's eye rash and acrodermatitis chronica atrophicans (“ACA”), the latter primarily caused by B. afzelii. Others include central nervous system Lyme neuroborreliosis (“LNB”), primarily caused by B. garinii, Lyme carditis of the heart, and Lyme arthritis (“LA”) in the joints, primarily caused by B. burgdorferi. Notably, the disease can go without specific signs and symptoms, and EM is only reported by approximately 30% of patients with (early) disseminated disease, and of that only 30-50% of patients diagnosed with LD recall a tick bite. Instead, patients may present with non-specific symptoms such as low-grade fever, myalgia, and arthralgia. In addition, although a typical EM might be easy to recognize, an EM does not manifest as a “classic” bull's eye rash in 30-40% of population. In daily practice, an EM is not as simple to diagnose as often suggested. Thus, a sensitive diagnostic test in the early stages of disease is needed that could accurately establish the diagnosis and so provide support for swift and proper treatment.

Serological testing that detects immune responses against the bacteria or bacterial proteins is currently the only diagnostic method approved by the U.S. FDA; it is the cornerstone of Lyme disease diagnostics, is required for early disseminated disease and late (disseminated) disease, and it is highly needed for early (localized) disease. It has been estimated that approximately 3.4 million serological tests are performed each year in the U.S. alone. In 1995, a two-tier testing method was implemented by the U.S. CDC. In this standard two-tiered testing (STTT) method, an enzyme immunosorbent assay (EIA) or immune-fluorescence assay (IFA) is employed as a first diagnostic test followed by separate IgM and IgG Western blots (WB) in the second step. However, the STTT algorithm is hampered by low sensitivity during early infection (30-50%), biased interpretation of bands obtained from WB, and misinterpretation of test results by health care workers as well as patients. Recently, several diagnostic tests with a modified two-tier test methodology (MTTT) with greater sensitivity were cleared by the U.S. FDA, which includes a separate EIA that replaces the WB in the second step of a STTT.

The most common protein targets for EIA are the VISE (for IgG) and OspC (for IgM). The use of the conserved C6 region of the VlsE in both steps of MTTTs and the 10-amino acid C-terminal peptide of OspC (“C10”) have significantly improved the specificity of the diagnosis method. These tests have been widely used in Europe since the conserved C6 region is present in most Borrelia genospecies. However, current tests still have low sensitivity in early infection, often use conventional antigens from old STTT methods, and are not yet used routinely in clinical and laboratory testing. Indeed, sensitivity of these tests in patients with early (localized) disease is 50% at best, and even for severe early disseminated LNB in Europe sensitivity is only 75%.

Beyond VISE (or C6) and OspC (or C10), only a handful of other Borrelia antigens have been considered for serodiagnostics, including FlaB (flagellin), BmpA (borrelial membrane protein A) and DbpA (decorin-binding protein A). Indeed, most innovations in Lyme disease diagnostics have been recycling known antigens in different assay formats and multiplex combinations. Combinations of these antigens, particularly inclusion of C10 IgM, have given better overall assay sensitivity and claims of >70% sensitivity for early LD. However, a ceiling may have been reached with the current selection of antigens. In other known tests, such as described in U.S. Pat. No. 5,985,595 an indirect cell based assay is employed for early stage detection of Borrelia. Here, binding characteristics of Borrelia or Borrelia peptides to polymorphonuclear leukocytes of a subject suspected of an infection are observed and correlated with infection with Borrelia. WO 2018/083491 teaches another indirect assay to test for the presence of Borrelia by detecting presence of release of Borrelia-specific bacteriophages from Borrelia. While conceptually relatively simple, such indirect tests are often time consuming, and specificity and sensitivity will typically be relatively low for early detection of B. afzelii.

In further known tests, as for example described in EP 3204404, the quantities of OspC antibodies and OspE antibodies are determined in a biological sample. Here, specific ratios of the antibodies are then correlated to early stage infection. Once more, while suitable for certain Borrelia species, specificity and sensitivity will typically be relatively low for early detection of B. afzelii. More recently, a diagnostic test for B. burgdorferi, which is the primary genospecies causing Lyme disease in the U.S., was approved by the U.S. FDA. However, that test does once more not directly translate to detection of B. afzelii, the most prevalent variant in Europe, which is responsible for approximately 80% of infections in Europe. In recent years, the incidence of Lyme disease has increased substantially. The World Health organization (WHO) and European Center for Disease Control and Prevention (ECDC) estimates 360,000 annual cases reported in the last two decades. The significant increase in Lyme disease incidence is predicted to be the consequence of multiple factors, including rapid climate change, that has allowed tick species to spread to higher altitudes and wider latitudes, making Lyme disease a significant vector-borne disease both in the U.S. and Europe. Unfortunately, despite the increasing need for Lyme disease diagnostic testing, and especially testing at early stages of disease, there are no known tests that would satisfy this need.

Thus, even though various diagnostic compositions and methods of detection of Lyme disease are known in the art, all or almost all of them suffer from several drawbacks, particularly where the detection is desired in early disease and/or where the pathogen is B. afzelii. Therefore, there remains a need for improved diagnostic compositions and methods of detection of Lyme disease.

### SUMMARY OF THE INVENTION

The inventive subject matter is directed to various compositions and methods of early detection of Lyme disease in which one or more antigenic proteins of B. afzelii are used to detect presence of antibodies in blood of a subject exposed to B. afzelii. Notably, antibodies binding these antigens are indicative of an early stage of Lyme disease of the subject. Indeed, using contemplated compositions and methods presented herein will enable detection of a B. afzelii infection with unprecedented sensitivity and specificity, typically in the early stage of Lyme disease.

In one aspect of the inventive subject matter, the inventors contemplate a test kit for detection of early Lyme disease, comprising a carrier coupled to an early-stage antigen of B. afzelii, wherein the antigen has at least 90% (or at least 95%, or at least 98%) identity to a protein sequence selected from the group consisting of SEQ ID NO:2, SEQ ID NO:4, and SEQ ID NO: 6, or an antigenic fragment thereof. Where desired, the antigen may have the protein sequence selected from the group consisting of SEQ ID NO:2, SEQ ID NO:4, and SEQ ID NO: 6, and/or an additional antigen of B. afzelii may be included, such as FlaB, P83/100, DbpA, DbpB, P66, VlsE, and/or OspC, or an antigenic fragment thereof.

In some embodiments, the carrier is a polymeric bead, a cellulosic membrane, or a wall of a microwell plate, and in further embodiments the antigen is a recombinant and at least partially purified antigen. As needed or otherwise desired, the antigen may further comprise a peptide portion for quantification (FLAG) or isolation (Tag).

Therefore, and viewed from a different perspective, the inventors also contemplate an antigen composition that includes an antigen of B. afzelii that is recognized in early Lyme disease. Preferably, the antigen has at least 90% (or at least 95%, or at least 98%, or 100%) identity to a protein sequence selected from the group consisting of SEQ ID NO:2, SEQ ID NO: 4, and SEQ ID NO:6, or an antigenic fragment thereof. Optionally, the antigen composition may further include one or more additional antigens of B. afzelii, and among other suitable antigens, FlaB, P83/100, DbpA, DbpB, P66, VlsE, and/or OspC, or an antigenic fragment thereof are particularly contemplated. For example, where the additional antigen is VlsE, the fragment may be the C6 fragment. Most typically, but not necessarily, the antigen and/or the additional antigen are recombinant and/or at least partially purified antigens.

Consequently, in another aspect of the inventive subject matter, the inventors contemplate an early-stage antigen of B. afzelii, for use in early detection of an infection/disease of a subject with B. afzelii, wherein the antigen has at least 90% (or at least 95%, or at least 98%, or 100%) identity to a protein sequence selected from the group consisting of SEQ ID NO: 2, SEQ ID NO:4, and SEQ ID NO:6, or an antigenic fragment thereof. As will be readily appreciated, the antigen may be a recombinant and/or at least partially purified antigen and may further include a peptide portion for quantification or isolation.

Therefore, the inventors also contemplate a method of detecting an early stage of infection/disease of a subject with B. afzelii that includes a step of contacting in vitro an early-stage antigen of B. afzelii with a blood sample of the subject. Preferably, the antigen has at least 90% (or at least 95%, or at least 98%, or 100%) identity to a protein sequence selected from the group consisting of SEQ ID NO:2, SEQ ID NO:4, and SEQ ID NO:6, or an antigenic fragment thereof. In a further step, binding of an antibody in the blood sample to the early-stage antigen is then detected.

As noted earlier, it is contemplated that the antigen may be a recombinant and/or at least partially purified antigen. Moreover, the antigen may also include a peptide portion for quantification or isolation. Most typically, binding of the antibody is detected via EIA, ELISA, CLIA, Luminex, Western blot, plasmon surface resonance, and/or spectroscopic detection. Where desired, contemplated methods may also include a step of detecting a binding event between an antibody in the blood and at least one more additional antigen of B. afzelii, wherein the additional antigen is selected form the group consisting of FlaB, P83/100, DbpA, DbpB, P66, VlsE, and OspC, or an antigenic fragment thereof. Notably, the detection has a sensitivity of at least 80% and a specificity of at least 90% at a cut-off value of equal or less than 0.6, and/or the early (acute) stage is no more than six weeks, no more than four weeks, or no more than 14 days post infection.

In a still further aspect of the inventive subject matter, the inventors contemplate a method of manufacturing a component for a test kit for detection of early stage Lyme disease and/or detection of infection of a subject with B. afzelii in an early stage of Lyme disease that includes a step of providing a nucleic acid or fragment thereof having at least 90% (or at least 95%, or at least 97%, or 100%) identity to a nucleic acid sequence selected from the group consisting of SEQ ID NO:1, SEQ ID NO:3, and SEQ ID NO:5, and a further step of recombinantly expressing the nucleic acid sequence or fragment thereof to thereby produce a recombinant protein that binds to an early-stage antigen of B. afzelii. In still another step, the recombinant protein is included into a test kit for detection of early-stage infection of a subject with B. afzelii.

Where desired, the nucleic acid further comprises a portion that encodes a peptide portion for quantification or isolation. Contemplated methods may also include a step of providing an additional nucleic acid encoding an additional antigen of B. afzelii, wherein the additional antigen is selected form the group consisting of FlaB, P83/100, DbpA, DbpB, P66, VlsE, and OspC, or an antigenic fragment thereof, a step of recombinantly expressing the additional nucleic acid, and a step of including the so expressed additional antigen into the test kit. As will be readily appreciated, the recombinant expression may be performed in a variety of manners, including expression in a bacterial expression system, and expression in an in vitro transcription/translation system.

In yet further aspects of the inventive subject matter, the inventors also contemplate a vaccine composition that includes a pharmaceutically acceptable carrier in combination with at least one antigen of B. afzelii or a nucleic acid encoding the antigen. In some embodiments, the antigen has at least 90% identity to an amino acid sequence selected from the group consisting of SEQ ID NO:2, SEQ ID NO:4, and SEQ ID NO:6, or an antigenic fragment thereof, and in further embodiments the nucleic acid has at least 90% identity to a nucleic acid sequence selected from the group consisting of SEQ ID NO:1, SEQ ID NO:3, and SEQ ID NO: 5, or portion thereof. Most typically, the portion of the nucleic acid encodes an antigenic fragment of SEQ ID NO:2, SEQ ID NO:4, or SEQ ID NO:6. As will be readily appreciated, the nucleic acid can be a DNA or an RNA transcript.

In some embodiments, the antigen has at least 95% identity to the amino acid sequence, or the antigen has an amino acid sequence selected from the group consisting of SEQ ID NO:2, SEQ ID NO:4, and SEQ ID NO:6. Similarly, in other embodiments, the nucleic acid sequence has at least 95% identity to SEQ ID NO:1, SEQ ID NO:3, or SEQ ID NO:5 or has the sequence of SEQ ID NO:1, SEQ ID NO:3, or SEQ ID NO:5. Where desired, the nucleic acid may be encapsulated in a lipid, and/or contemplated vaccine compositions may further comprise an adjuvant.

Various objects, features, aspects, and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 is a schematic illustration of an experimental workflow to obtain blood samples from mice infected with B. afzelii.

FIG. 2 is a heat map for IgG and IgM responses across different stages of mice infected with B. afzelii.

FIG. 3A is a heat map for IgG and IgM responses across different stages of human infected with B. afzelii.

FIG. 3B is a graph depicting IgG responses to selected antigens across different stages of human infected with B. afzelii.

FIG. 4 is a schematic overview of an experimental workflow according to the inventive subject matter.

FIG. 5A is an ROC plot for a first new antigen for early detection of Lyme disease.

FIG. 5B is an ROC plot for a second new antigen for early detection of Lyme disease.

FIG. 5C is an ROC plot for a third new antigen for early detection of Lyme disease.

FIG. 5D is an ROC plot for a known antigen for detection of Lyme disease.

FIG. 6 is an ROC plot for a combination of the first new antigen and a known antigen for detection of Lyme disease.

### DETAILED DESCRIPTION

The inventors have discovered various antigens that exhibit unexpected and unprecedented high sensitivity and selectivity for detection of Lyme disease, even where the Lyme disease is in an early stage. Such discovery is particularly remarkable as each of these antigens not only have superior detection characteristics for early Lyme disease as compared to heretofore known antigens, but also in that these antigens are suitable for detection of Lyme disease across all stages of Lyme disease.

In this context it should be noted that currently known tests for Lyme disease detection are typically only reliable in the late disease stage while earlier stages such as early acute and early convalescent stages are typically not reliably diagnosed using known antigens such as FlaB, P83/100, DbpA, DbpB, P66, OspC, and VISE (and its C6 fragment). Viewed from a different perspective, it should be recognized that the new antigens presented herein will be particularly useful to detect early Lyme disease. Therefore, suitable antigens might allow detection of early Lyme disease and/or infection with B. afzelii within the early acute (≤6 wks) and the convalescent (≥6 wks) phase of the disease. Most typically, but not necessarily, such detection with follow conventional test protocols well known in the art in which the currently known antigens are replaced or supplemented with the newly discovered antigens presented herein.

Among other possible antigens, particularly preferred antigens are D0016 (encoded by nucleic acid sequence SEQ ID NO:1 and having protein sequence SEQ ID NO:2), A0029 (encoded by nucleic acid sequence SEQ ID NO: 3 and having protein sequence SEQ ID NO:4), and A0001 (encoded by nucleic acid sequence SEQ ID NO: 5 and having protein sequence SEQ ID NO: 6) and all antigenic and/or diagnostic fragments thereof as is described in more detail below. Consequently, the sequences presented herein are especially contemplated for diagnostic use, and especially diagnostic use for detection of early Lyme disease. As used herein and unless expressly stated otherwise, the term “early Lyme disease” or “early stage of Lyme disease” refers to the early acute stage that is no more than six weeks after onset of manifestation or suspected tick bite, as well as the convalescent-phase early Lyme disease that may be greater than six weeks after onset of manifestation or suspected tick bite. In such stages EM may or may not be present.

With respect to the antigens presented herein it should be recognized that while specific sequences are disclosed, numerous modifications of these sequences are also deemed suitable for use herein. For example, each of the sequences of SEQ ID NO: 1-6 need not be exactly as described in the sequence listing but may be altered in response to one or more considerations. For example, nucleic acid sequences may be modified to adapt to a specific codon usage of a cell where the sequence is to be expressed. Indeed, where the nucleic acid sequences are expressed in a cell based (e.g., in E. coli or P. pasteuris) or cell free system (e.g., in an in vitro transcription/translation system) one or more regulatory sequences may be provided in functional context to drive and/or control expression of the recombinant protein product. In still other contemplated modifications, the nucleic acid sequence may further contain (typically in frame) a nucleic acid sequence that encodes for a flag and/or a tag peptide to facilitate quantification of the recombinant product and/or facilitate isolation of the recombinant product.

Still further, it should be noted that the antigen need not be a full-length antigen as exemplarily shown in SEQ ID NOs: 1-6, but that the sequences may be truncated on the 5′- and/or 3′-end or the N- and/or C-terminus. Especially preferred truncated versions will include at least the antigenic portion of the antigen (the portion or domain to which the antibodies bind) typically be preferred where the antigenic. Therefore, contemplated truncated sequences may have a length of between about 12 amino acids to 50 amino acids, or between 25-70 amino acids, and in some cases even longer. Moreover, it should be noted that the antigen sequences contemplated herein may also have one or more amino acid substitutions, insertions, and/or deletions so long as such modifications will not adversely affect binding of an antibody to the antigen. Therefore, contemplated antigens may have at least 90% identity, or at least 92% identity, or at least 94% identity or at least 96% identity or at least 98% identity to a protein sequence selected from the group consisting of SEQ ID NO:2, SEQ ID NO:4, and SEQ ID NO: 6. Of course, it should be recognized that corresponding changes may also be present in the nucleic acids encoding the antigens presented herein. Therefore, contemplated nucleic acids encoding the antigens may have at least 90% identity, or at least 92% identity, or at least 94% identity or at least 96% identity or at least 98% identity to a nucleic acid sequence selected from the group consisting of SEQ ID NO: 1, SEQ ID NO:3, and SEQ ID NO:5.

In further contemplated embodiments, the antigen protein may be isolated or enriched from an B. afzelii cell and/or cell extract. Regardless of the manner of production, it is contemplated that the antigen protein may be part of a crude extract or transcription/translation mix, or that the antigen protein may be at least partially purified (e.g., using a his-tag).

Most typically, the antigen will be immobilized or coupled (e.g., covalently or via an affinity binder) to a solid phase that is then brough in contact with a biological fluid (typically blood or serum or plasma) of a subject infected with B. afzelii. For example, suitable solid include walls of a multiwell plate, polymeric carriers, or binders (e.g., plastic beads, optionally color coded), cellulosic membranes or coatings such as nitrocellulose membranes, functionalized glass surfaces, etc. Once the antigen is immobilized or coupled to the solid phase, the biological fluid is then brought in contact with the antigen and any antibody in the biological fluid that binds specifically to the antigen will be retained on the solid phase while other components can be washed off or otherwise removed. Detection of the bound antibody then follows standard protocol well known in the art. Viewed from a different perspective, it should be appreciated that the antigens and variants and fragments thereof presented herein (especially having amino acid sequence SEQ ID NO:2, SEQ ID NO:4, and/or SEQ ID NO:6) can be used in the detection of early Lyme disease (and as is shown in more detail below, also in all other stages of Lyme disease, rendering these antigens pan-stage specific antigens) by detecting IgG and/or IgM antibodies in blood or serum of an individual infected with B. afzelii. Consequently, it should be noted that among other detection methods colorimetric, radiometric, spectrophotometric methods as well as surface plasmon resonance are all deemed suitable for use herein. Viewed from a different perspective, antibody detection from a patient sample may include EIA, ELISA tests, CLIA, Luminex, Western blot, SPR, RIA, etc. Of course, it should be noted that contemplated antigens are equally useful for Tier 1 and Tier 2 tests.

While the European predominant species B. afzelii is especially contemplated herein, other species and subspecies are also deemed suitable for use, including B. burgdorferi and B. garinii. In such cases, it is contemplated that the sequence homologs for B. burgdorferi and B. garinii of the antigen sequences presented herein are also deemed appropriate. Moreover, it is contemplated that the antigens of the inventive subject matter may also be combined in a test with currently known antigens to improve sensitivity and/or selectivity. For example, contemplated additional antigens include one or more of FlaB, P83/100, DbpA, DbpB, P66, VlsE, and OspC, or an antigenic (antibody-binding) fragment thereof. For example, where the additional antigen is VlsE, a preferred fragment of VISE is C6.

Consequently, it should be recognized that the inventors contemplate test kits and antigen compositions comprising the antigens presented herein, preferably in a format that allows for testing of a biological fluid (and especially blood or serum or plasma) for the presence of antibodies that bind to such antigens. Most typically, the antibodies will be IgG class antibodies, but other classes are also deemed appropriate, including IgM and IgA. Thus, these antigens will also be especially suitable for use in early detection of an infection of a subject with B. afzelii.

While the antigens presented herein are deemed especially suitable for diagnostic use, it should be appreciated that the antigens may also be useful for the manufacture of a vaccine composition. Suitable vaccine compositions will preferably comprise at least one of the antigens presented herein, and the antigens may be included as peptide antigens or in a nucleic acid vaccine format in which the nucleic acid encodes one or more of the antigens. As should be readily recognized, the nucleic acid may be in form of a DNA or an RNA. Moreover, it is contemplated that the antigen(s) or nucleic acid encoding the antigen, need not be identical with the antigens presented herein, but that suitable amino acid or nucleic acid sequences may have a sequence identity of at least 90%, or at least 92%, or at least 94%, or at least 96%, or at least 98% to the sequences of SEQ ID NOs: 1-6.

For example, where contemplated vaccine compositions are protein based vaccines, the vaccine composition may include one or more of proteins having the amino acid sequence of SEQ ID NO:2, SEQ ID NO:4, and/or SEQ ID NO:6 (or an antigenic fragment thereof). Most typically, such protein based vaccines will further include an adjuvant and will be formulated for injection (e.g., intramuscular or subdermal). On the other hand, where the vaccine composition is formulated as a nucleic acid vaccine, the vaccine may include a DNA or RNA form of the nucleic acid sequences of SEQ ID NO:1, SEQ ID NO:3, and/or SEQ ID NO:5 (or portion thereof where the portion encodes an immunogenic fragment of SEQ ID NO:2, SEQ ID NO: 4, and/or SEQ ID NO:6. Most typically, these nucleic acid sequences will be coupled to suitable control sequences that enable in vivo transcription/translation to so give rise to the corresponding peptide sequences. Moreover, it is contemplated that the nucleic acid sequences will be encapsulated into a lipid carrier/vesicle to so allow formulation into an injectable vaccine formulation. There are numerous compositions and methods known in the art to make protein and nucleic acid vaccines, and all known manners are deemed suitable for use herein.

### EXAMPLES

The first phase for a serodiagnostic assay for European Lyme disease (caused by B. afzelii) was based on the development of a B. afzelii proteome microarray for quantification of IgG and IgM binding to known and unknown B. afzelii antigens in sera from (a) mice experimentally infected with B. afzelii through tick-bite and (b) patients with varying stages of LD and in healthy control samples.

Development of a B. afzelii proteome microarray: Using a known proteome microarray development pathway (see e.g., WO 2008/140478; URL: antigendiscovery.com/adi-proteome-microarray-technology), the B. afzelii BO23 genome was analyzed for cloning design of protein-coding genes. In short, each protein, epitope, domain, or protein fragment's gene sequence was amplified by PCR and inserted into the vector pXT7 by recombination in E. coli to establish a library of partial or complete coding DNA sequences using genomic template Borrelia afzelii PKo (amplified from BO23). Clones were sequence verified, expressed using a coupled E. coli cell-free in vitro transcription and translation (IVTT) system and spotted onto nitrocellulose-coated microarray slides. Protein expression was checked by probing slides with anti-His and anti-HA tag monoclonal antibodies, which detect the N-term and C-term tags on each IVTT protein, respectively. The B. afzelii full-proteome microarray contained 1,308 full-length or fragmented proteins representing 1,283 of the 1,365 genes in B. afzelii BO23 (94% coverage).

Microarrays were probed with sera and antibody binding detected by incubation with fluorochrome-conjugated goat anti-human IgG or IgA or IgM (Jackson ImmunoResearch, West Grove, PA, USA or Bethyl Laboratories, Inc., Montgomery, TX, USA). Slides were scanned on a GenePix 4300A High-Resolution Microarray Scanner (Molecular Devices, Sunnyvale, CA, USA), and raw spot and local background fluorescence intensities, spot annotations and sample phenotypes were imported and merged in R (R Core Team, 2017), in which all subsequent procedures were performed.

Raw signal acquisition: Probed microarrays (slides) were scanned using a GenePix 4300A high-resolution microarray scanner (Molecular Devices, Sunnyvale, CA), and an image file (.tiff) was saved for each array using GenePix pro 7 software. The signals in the scanned images were quantified using the Mapix software (Innopsys) autogridding feature. For this process, two input files were required: (i) a. gal file that defines the array and subarray layout, and (ii) the .tiff image file for an array. Once the autogridding was complete, the overlays of the mapped array, subarray, and individual spot locations were shown in the graphical user interface (GUI). If the automatic gridding fails to map to the correct positions, the mapping can be manually adjusted using the GUI. Once the gridding were confirmed to be correct, the array spots were quantified and saved to an output .gpr file. For each spot on the slide, the .gpr file contained the foreground intensity (median of pixels inside the circle defining the spot) and local background intensity (median of pixels just outside the circle defining the spot). The final raw intensity was the foreground intensity minus the local background intensity. The raw signals were automatically extracted and saved as .csv files in data matrix format, with array spots as rows and samples as columns, using R (URL: www.R-project.org).

Proteome microarray data normalization: First, raw values were transformed using the base 2 logarithm. Next, the data set was normalized to remove systematic effects by subtracting the median signal intensity of the IVTT control spots for each sample. Since the IVTT control spots carry not only the chip, sample, and batch-level systematic effects, but also antibody background reactivity to the IVTT system, this procedure normalizes the data and provides a relative measure of the specific antibody binding versus the nonspecific antibody binding to the IVTT controls. With the normalized data, a value of 0.0 means that the intensity is no different than that of the IVTT controls, and a value of 1.0 indicates a doubling with respect to IVTT control spots.

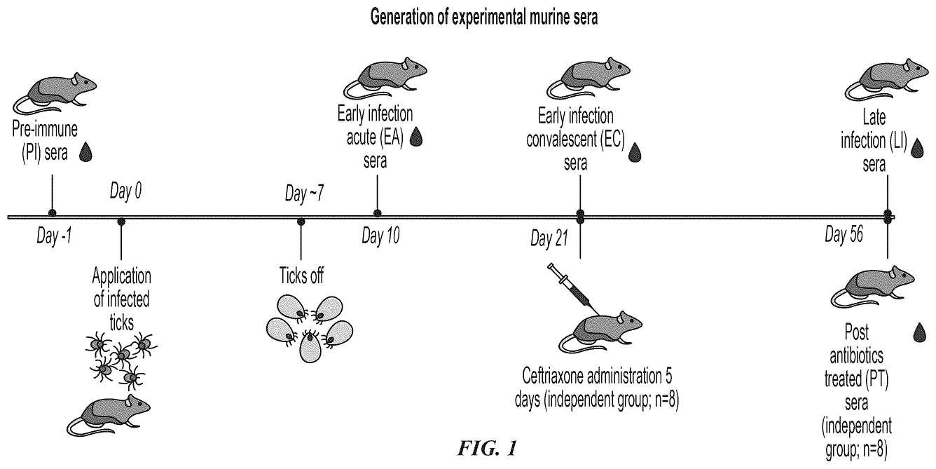

Discovery of new diagnostic antigens for a simple single-tier serodiagnostic assay for detection of both early and late stages of Lyme disease: The B. afzelii full-proteome microarray was probed with two sets of serum samples, a mouse set and human set. The study design of the mouse samples is shown in FIG. 1, and details of the patient population for the human samples is shown in Table 1.

TABLE 1
Selection of human serum samples in discovery/preliminary study

| | | Number of |
|---|---|---|
| Group | Sample timeline | samples |
| Healthy blood donors (controls) | Not applicable | 50 |
| Early disease (Erythema migrans—EM) * | Acute and | 49 |
| | convalescent ^{‡} | |
| Late disease (Acrodermatitis chronica | Convalescent | 25 |
| atrophicans—ACA) ^{†} | | |
| Post-treatment Lyme disease syndrome | Convalescent | 25 |
| (PTLDS) ^{¥} | | |

| | |
|---|---|
| Total number of samples | 149 |

* PCR and/or culture confirmed samples
^{‡} Acute ≤6 weeks and convalescent ≥6 weeks after onset of manifestation
^{†} PCR and/or culture confirmed samples
^{¥} Confirmed based on criteria defined by Coumou J. et al 2014

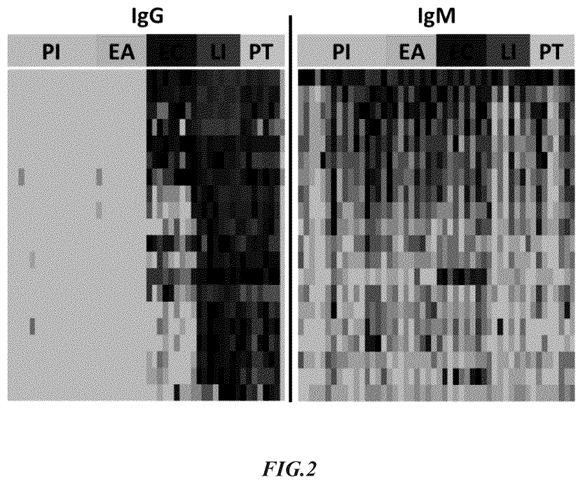

In the first study, sera were derived from mice experimentally infected with B. afzelii by placing infectious I. ricinus ticks (harboring B. afzelii) for ˜7 days and sampled during follow-up (FIG. 1). Time points were: “PI” pre-infection or immune (n=16), “EA” early acute phase (day 10) (n=8), “EC” early convalescent (day 21) (n=8), “LI” late infection (day 56 in untreated mice) (n=8), and “PT” post-treatment (day 56 in antibiotics treated mice—independent group, n=8). A total of 111 B. afzelii proteins were recognized by IgG and 282 proteins recognized by IgM in at least one mouse (data not shown). FIG. 2 depicts the antibody profile of 20 most reactive B. afzelii proteins in the mouse experimental infections study. The heatmaps show IgG (left) and IgM binding (right) to 20 B. afzelii antigens (rows) with highest means on a color scale from green to red. Each column represents a mouse sample and is grouped by disease phase. PI: pre-infection; EA: early acute; EC: early convalescent; LI: late infection; PT: post-treatment.

Notably, among the IgM-reactive proteins, one antigen was significantly differentially reactive between PI and EC, and 15 antigens between PI and LI after correction for the false discovery rate, as can be seen from Table 2. No antigens were significantly differentially reactive in PI vs. EA and in PI vs. PT, however, the significant antigen between PI and EC was borderline significant between PI and EA (Table 2). For IgG, no antigens were different in PI vs. EA unsurprisingly, but 58 were different in PI vs. EC, 70 for PI vs. LI, 73 for PI vs. PT and 35 for LI vs. PT.

TABLE 2

| Mouse | IgG | IgM | Human | IgG | IgM |
|---|---|---|---|---|---|
| PI vs. EA | 0 | 0 | CO vs. EA | 3 | 0 |
| PI vs. EC | 58 | 1 | CO vs. EC | 3 | 1 |
| PI vs. LI | 70 | 15 | CO vs. LD | 92 | 21 |
| PI vs. PT | 73 | 0 | CO vs. PTLDS | 2 | 0 |
| LI vs. PT | 35 | 0 | LD vs. PTLDS | 65 | 5 |

PI: pre-infection;
EA: early acute;
EC: early convalescent;
LI: late infection;
PT: post treatment;
CO: control;
LD: late disease;
PTLDS: post treatment Lyme disease syndrome

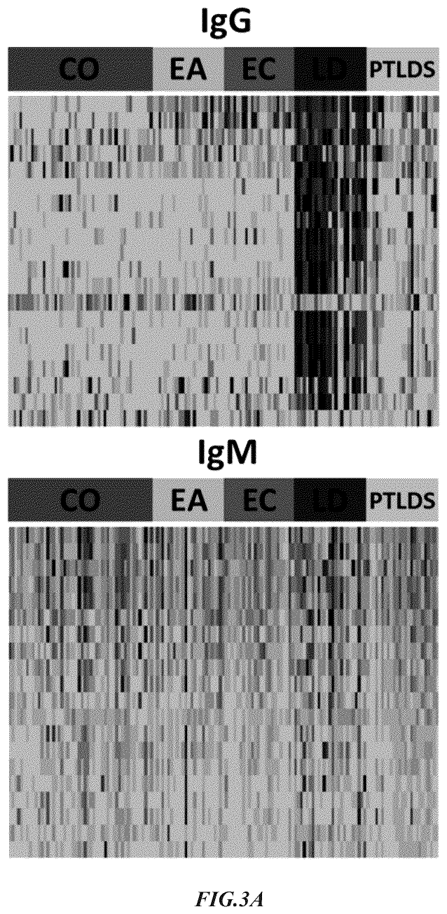

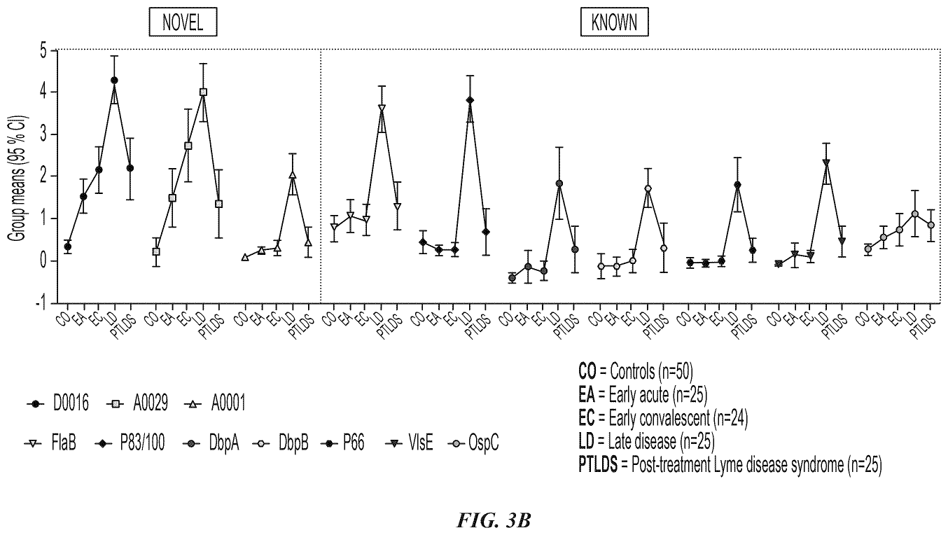

In the second study, human sera from healthy controls (“CO”) were compared with the following cross-sections of patients: “EA” early acute and “EC” early convalescent disease (erythema migrans), “LD” late disease (acrodermatitis chronica atrophicans), and “PTLDS” patients. In humans, a total of 422 B. afzelii proteins were recognized by IgG and 410 proteins recognized by IgM in at least 10% of the exposed population as is shown in FIG. 3A. Here, the antibody profile of 20 most reactive B. afzelii proteins is shown in human patient samples and disease phase profiles of top diagnostic antigens. The heatmaps show IgG binding (top) and IgM binding (bottom) to 20 B. afzelii antigens (rows) with highest means on a color scale from green to red. Each column represents a human sample and is grouped by disease phase. CO: healthy controls; EA: early acute; EC: early convalescent; LD: late disease; PTLDS: post treatment Lyme disease syndrome. Notably, For IgG, three antigens were differentially reactive between controls and early disease groups after correction for the false discovery rate as is depicted in FIG. 3B. Here, the line graphs show IgG responses to the three top novel diagnostic antigens (left) and a set of known antigens (right). These three antigens were D0016 (having nucleic acid sequence SEQ ID NO: 1 and protein sequence SEQ ID NO:2), A0029 (having nucleic acid sequence SEQ ID NO: 3 and protein sequence SEQ ID NO:4), and A0001 (having nucleic acid sequence SEQ ID NO: 5 and protein sequence SEQ ID NO:6). Group means and 95% confidence intervals are plotted for the disease stages (x-axis, with sample size) shown in the key at the top right of the graph. Only one antigen was differentially reactive in the same group comparisons for IgM (p=0.01 before adjustment in CO vs. EA). Notably, these were all novel antigens for B. afzelii serodiagnostics. For late disease, 92 antigens had significant differences from controls for IgG and 21 for IgM. In PTLDS patients, 46 antigens had significant differences from controls for IgG and 16 for IgM.

Among the many IgG- and IgM-reactive antigens identified and differential reactivities discovered, several antigens bear promise as a multi-antigen biomarker for detection of both early and late European Lyme disease. Several known antigens and many novel antigens can clearly differentiate healthy patients from late Lyme disease patients, but selected antigens discovered here may significantly increase sensitivity for diagnosis of early Lyme disease for which the currently known antigens cannot. Notably, and as is shown in more detail below, none of the known diagnostic antigens have the profile seen in early Lyme disease (FIG. 3B). Moreover, the new antigens discovered here also exhibit a remarkable sensitivity and specificity as compared to heretofore known antigens. Table 3 below exemplarily illustrates the performance of two of the three new antigens versus a known antigen (VlsE) in distinguishing Lyme disease patients at various stages of disease from uninfected controls by Receiver Operating Characteristics (ROC) area under the curve (AUC).

TABLE 3
AUC values of D0016 and A0029 antigens
as compared to AC0019 (VlsE)

| | | CO vs. | CO vs. | CO vs. |
|---|---|---|---|---|
| SEQ ID | Description | EA | EC | LD |
| BafPKo_D0016 | fibronectin-binding | 0.90 | 0.94 | 0.99 |
| | protein | | | |
| BafPKo_A0029 | hypothetical protein | 0.82 | 0.89 | 0.95 |
| BafPKo_AC0019 | Outer surface | 0.65 | 0.67 | 0.99 |
| | protein VlsE | | | |

Abbreviations: CO, Control;
EA, Early Acute;
EC, Early Convalescent;
LD, Late Disease

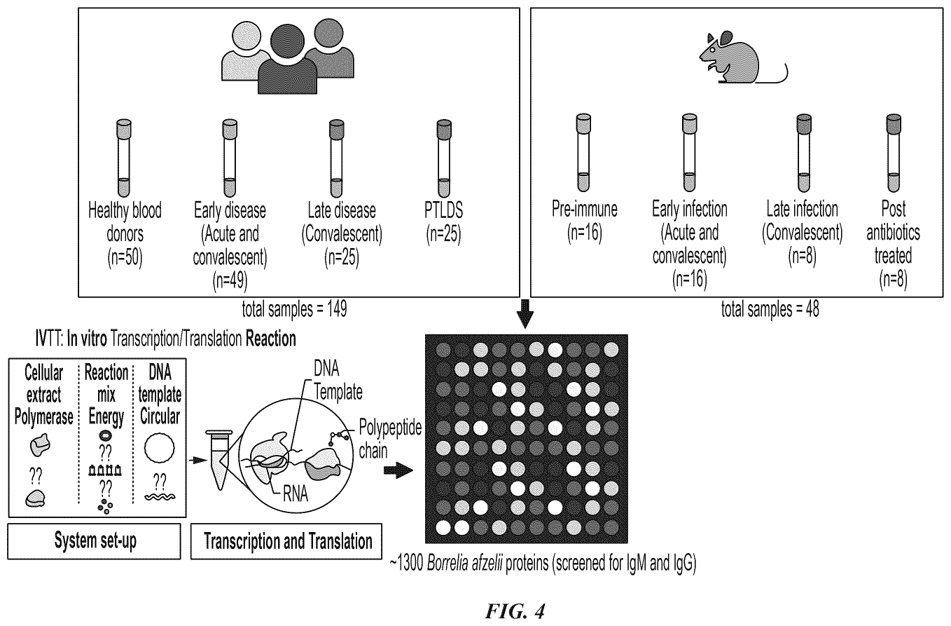

As can be readily taken form the Table above and FIG. 3B, the new antigens had in addition to the ability of early-stage detection also the ability to detect all other stages with high sensitivity and specificity. FIG. 4 exemplarily and schematically depicts the above discovery process of these new antigens.

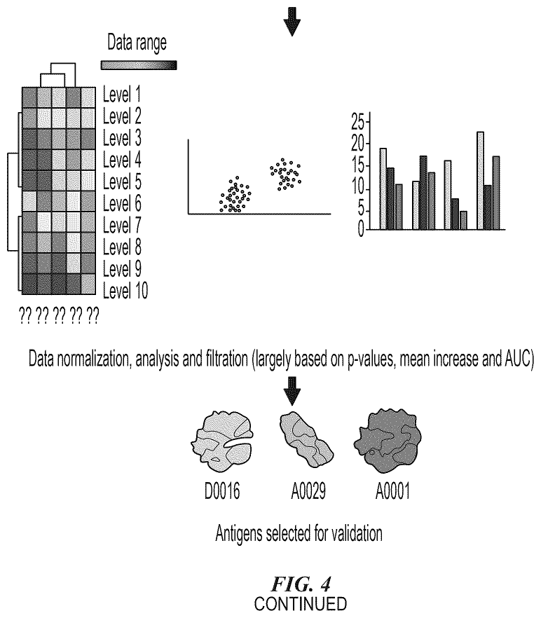

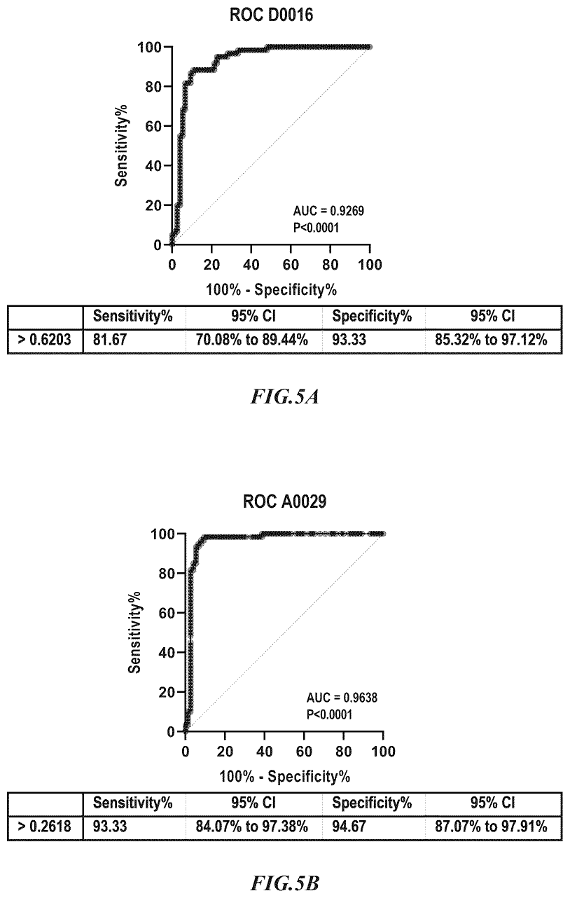

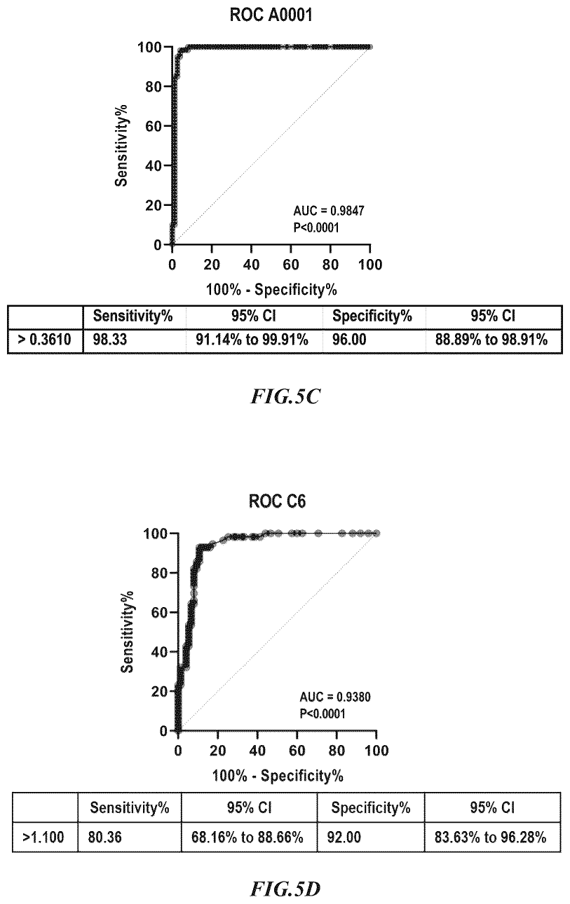

In still further experiments, the inventors set out to determine sensitivity and specificity for each of the newly discovered antigens in an independent sample set (Table 4), and exemplary results are shown in in FIG. 5A (for D0016), FIG. 5B (for A0029), and FIG. 5C (for A0001). As can be seen for the results in the graphs of FIG. 5A-C, each of the three antigens had a remarkable sensitivity and specificity that surpassed the corresponding results in FIG. 5D. Interestingly, while D0016 had a higher sensitivity and specificity as compared to the currently known C6 peptide, and as such would be advantageous as a standalone antigen, the data for D0016 were inferior to those for A0029 and A0001. Thus, A0001 was the most promising test candidate, at least with respect to a single antigen test.

TABLE 4
Selection of human serum samples for preliminary
sensitivity and specificity analysis)

| | | Number of |
|---|---|---|
| Group | Sample timeline | samples |
| Healthy blood donors (controls) | Not applicable | 75 |
| Early disease (Erythema migrans—EM) * | Acute and | 60 |
| | convalescent ^{‡} | |

| | |
|---|---|
| Total number of samples | 135 |

* Well-defined, PCR and/or culture confirmed samples
^{‡} Acute ≤6 weeks and convalescent ≥6 weeks after onset of manifestation

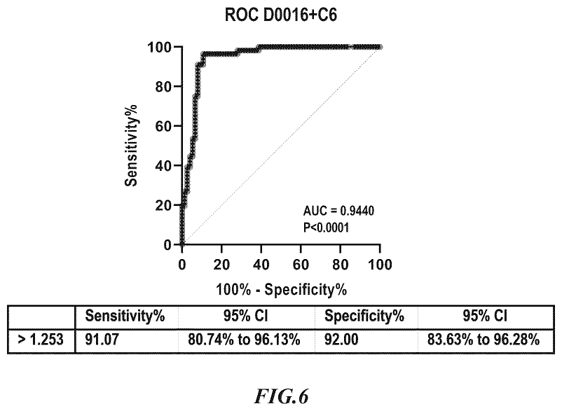

The inventors further evaluated if D0016 would improve sensitivity in a 1 Tier (ELISA alone) and a 2 Tier (ELISA followed by Western blot) test system. In short, when adding data derived from D0016 and C6 independently, a cumulative 9.47% increase in sensitivity (91.07% vs 81.6%) was observed while maintaining the same specificity level (92%). As a reference, when C6 peptide alone (well-established and routinely used) was used in a STTT set up, sensitivity was 38% and specificity was 97% (data not shown). Therefore, D0016 provided a marked advantage in sensitivity as compared to testing with the C6 peptide (alone or as a STTT set up). FIG. 6 depicts exemplary results for the combination of D0016 with C6.

In view of the above, it should therefore be recognized that D0016, A0029, and A0001 present three novel antigens with high immune responses for all stages, and especially at an early disease stage. Indeed, all three antigens had significantly high IgG responses in samples from donors with early disease as compared to healthy blood donors. Moreover, A0029 and A0001 demonstrated very high sensitivity (93.3% and 98.3%) and specificity (94.6% and 96%) as compared to the current C6 test peptide (80.3% and 92% of sensitivity and specificity; implementing the cut-off value routinely used in commercial assays) and D0016 exhibited a superior detection profile as compared to the C6 peptide alone.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. The recitation of ranges of values herein is merely intended to serve as a shorthand method of referring individually to each separate value falling within the range. Unless otherwise indicated herein, each individual value is incorporated into the specification as if it were individually recited herein.

As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). It should further be noted that the terms “prognosing” or “predicting” a condition, a susceptibility for development of a disease, or a response to an intended treatment is meant to cover the act of predicting or the prediction (but not treatment or diagnosis of) the condition, susceptibility and/or response, including the rate of progression, improvement, and/or duration of the condition in a subject.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As also used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

What is claimed is:

1. A test kit for detection of early Lyme disease, comprising a carrier coupled to an early-stage antigen of B. afzelii, wherein the antigen has at least 90% identity to a protein sequence selected from the group consisting of SEQ ID NO:2, SEQ ID NO:4, and SEQ ID NO: 6, or an antigenic fragment thereof.

2. The test kit of claim 1, wherein the antigen has at least 95% identity to the protein sequence.

3. The test kit of claim 1, wherein the antigen has at least 98% identity to the protein sequence.

4. The test kit of claim 1, wherein the antigen has the protein sequence selected from the group consisting of SEQ ID NO:2, SEQ ID NO:4, and SEQ ID NO:6.

5. The test kit of any one of the preceding claims, further comprising an additional antigen of B. afzelii, wherein the additional antigen is selected form the group consisting of FlaB, P83/100, DbpA, DbpB, P66, VlsE, and OspC, or an antigenic fragment thereof.

6. The test kit of claim 1, wherein the carrier is a polymeric bead, a cellulosic membrane, or a wall of a microwell plate.

7. The test kit of claim 1, the antigen is a recombinant and at least partially purified antigen.

8. The test kit of claim 1, wherein the antigen further comprises a peptide portion for quantification or isolation.

9. The test kit of claim 1, further comprising an additional antigen of B. afzelii, wherein the additional antigen is selected form the group consisting of FlaB, P83/100, DbpA, DbpB, P66, VlsE, and OspC, or an antigenic fragment thereof.

10. The test kit of claim 1, wherein the carrier is a polymeric bead, a cellulosic membrane, or a wall of a microwell plate.

11. The test kit of claim 1, wherein the antigen is a recombinant and at least partially purified antigen.

12. The test kit of claim 1, wherein the antigen further comprises a peptide portion for quantification or isolation.

13. An antigen composition, comprising:
an early-stage antigen of B. afzelii, wherein the antigen has at least 90% identity to a protein sequence selected from the group consisting of SEQ ID NO:2, SEQ ID NO: 4, and SEQ ID NO:6, or an antigenic fragment thereof; and
optionally an additional antigen of B. afzelii, wherein the additional antigen is selected form the group consisting of FlaB, P83/100, DbpA, DbpB, P66, VlsE, and OspC, or an antigenic fragment thereof.

14. The antigen composition of claim 13, wherein the antigen has at least 95% identity to the protein sequence.

15. The antigen composition of claim 13, wherein the antigen has at least 98% identity to the protein sequence.

16. The antigen composition of claim 13, wherein the antigen has the protein sequence selected from the group consisting of SEQ ID NO:2, SEQ ID NO:4, and SEQ ID NO:6.

17. The antigen composition of any one of claims 13-16, further comprising at least one additional antigen.

18. The antigen composition of claim 17, wherein the additional antigen is VlsE or a fragment thereof.

19. The antigen composition of any one of claims 13-18, wherein the antigen and/or the additional antigen is a recombinant and at least partially purified antigen.

20. The antigen composition of claim 13, further comprising at least one additional antigen.

21. The antigen composition of claim 20, wherein the additional antigen is VlsE or a fragment thereof.

22. The antigen composition of claim 13, wherein the antigen and/or the additional antigen is a recombinant and at least partially purified antigen.

23. An antigen of B. afzelii for use in detection of early Lyme disease and/or infection of a subject with B. afzelii, wherein the antigen has at least 90% identity to a protein sequence selected from the group consisting of SEQ ID NO:2, SEQ ID NO:4, and SEQ ID NO:6, or an antigenic fragment thereof.

24. The early-stage antigen of claim 23, wherein the antigen has at least 95% identity to the protein sequence.

25. The early-stage antigen of claim 23, wherein the antigen has at least 98% identity to the protein sequence.

26. The early-stage antigen of claim 23, wherein the antigen has the protein sequence selected from the group consisting of SEQ ID NO:2, SEQ ID NO:4, and SEQ ID NO:6.

27. The early-stage antigen of any one of claims 23-26, wherein the antigen is a recombinant and at least partially purified antigen and optionally comprises a peptide portion for quantification or isolation.

28. A method of detecting early Lyme disease and/or an early stage of infection of a subject with B. afzelii, comprising:
contacting in vitro an antigen of B. afzelii with a blood sample of the subject;
wherein the antigen has at least 90% identity to a protein sequence selected from the group consisting of SEQ ID NO:2, SEQ ID NO:4, and SEQ ID NO:6, or an antigenic fragment thereof; and
detecting binding of an antibody in the blood sample to the antigen.

29. The method of claim 28, wherein the antigen has at least 95% identity to the protein sequence.

30. The method of claim 28, wherein the antigen has at least 98% identity to the protein sequence.

31. The method of claim 28, wherein the antigen has the protein sequence selected from the group consisting of SEQ ID NO:2, SEQ ID NO:4, and SEQ ID NO:6.

32. The method of claim 28, wherein the antigen is a recombinant and at least partially purified antigen and optionally comprises a peptide portion for quantification or isolation.

33. The method of any one of claims 28-32, wherein binding of the antibody is detected via ELISA, Western blot, plasmon surface resonance, or spectroscopic detection.

34. The method of any one of claims 28-32, further comprising a step of detecting a binding event between an antibody in the blood and an additional antigen of B. afzelii, wherein the additional antigen is selected form the group consisting of FlaB, P83/100, DbpA, DbpB, P66, VlsE, and OspC, or an antigenic fragment thereof.

35. The method of any one of claims 28-32, wherein detection has a sensitivity of at least 80% and a specificity of at least 90% at a cut-off value of equal or less than 0.6.

36. The method of any one of claims 28-32, wherein the blood sample is obtained from the subject within an early acute stage or convalescent stage of Lyme disease.

37. A method of manufacturing a component for a test kit for detection of early-stage infection of a subject with B. afzelii or early disease of a subject, comprising:
providing a nucleic acid or fragment thereof having at least 90% identity to a nucleic acid sequence selected from the group consisting of SEQ ID NO:1, SEQ ID NO: 3, and SEQ ID NO:5;
recombinantly expressing the nucleic acid sequence or fragment thereof to thereby produce a recombinant protein that binds to an early-stage antigen of B. afzelii; and
including the recombinant protein into a test kit for detection of early-stage infection of a subject with B. afzelii.

38. The method of claim 37, wherein the nucleic acid or fragment thereof has at least 95% identity.

39. The method of claim 37, wherein the nucleic acid or fragment thereof has at least 97% identity.

40. The method of claim 37, wherein the nucleic acid or fragment thereof nucleic acid or fragment thereof has the nucleic acid sequence selected from the group consisting of SEQ ID NO: 1, SEQ ID NO:3, and SEQ ID NO:5.

41. The method of any one of claims 37-40, wherein the nucleic acid further comprises a portion that encodes a peptide portion for quantification or isolation.

42. The method of any one of claims 37-40, further comprising providing an additional nucleic acid encoding an additional antigen of B. afzelii, wherein the additional antigen is selected form the group consisting of FlaB, P83/100, DbpA, DbpB, P66, VlsE, and OspC, or an antigenic fragment thereof, recombinantly expressing the additional nucleic acid, and including the so expressed additional antigen into the test kit.

43. The method of any one of claims 37-40, wherein the recombinant expression is performed in a bacterial expression system or in an in vitro transcription/translation system.

44. The method of claim 37, further comprising providing an additional nucleic acid encoding an additional antigen of B. afzelii, wherein the additional antigen is selected form the group consisting of FlaB, P83/100, DbpA, DbpB, P66, VlsE, and OspC, or an antigenic fragment thereof, recombinantly expressing the additional nucleic acid, and including the so expressed additional antigen into the test kit.

45. The method of claim 37, wherein the recombinant expression is performed in a bacterial expression system or in an in vitro transcription/translation system.

46. A vaccine composition, comprising:
a pharmaceutically acceptable carrier in combination with at least one antigen of B. afzelii or a nucleic acid encoding the antigen;
wherein the antigen has at least 90% identity to an amino acid sequence selected from the group consisting of SEQ ID NO:2, SEQ ID NO:4, and SEQ ID NO:6, or an antigenic fragment thereof; and
wherein the nucleic acid has at least 90% identity to a nucleic acid sequence selected from the group consisting of SEQ ID NO:1, SEQ ID NO:3, and SEQ ID NO:5, or portion thereof, wherein the portion encodes an antigenic fragment of SEQ ID NO: 2, SEQ ID NO:4, or SEQ ID NO:6, or wherein the nucleic acid is an RNA transcript of the nucleic acid sequence or portion thereof.

47. The vaccine composition of claim 46, wherein the antigen has at least 95% identity to the amino acid sequence.

48. The vaccine composition of claim 46, wherein the antigen has an amino acid sequence selected from the group consisting of SEQ ID NO:2, SEQ ID NO:4, and SEQ ID NO:6.

49. The vaccine composition of claim 46, wherein the nucleic acid sequence has at least 95% identity to SEQ ID NO:1, SEQ ID NO:3, or SEQ ID NO:5.

50. The vaccine composition of claim 46, wherein the nucleic acid sequence has at SEQ ID NO: 1, SEQ ID NO:3, or SEQ ID NO:5.

51. The vaccine composition of any one of claims 49-50, wherein the nucleic acid is the RNA transcript.

52. The vaccine composition of any one of claims 49-50, wherein the nucleic acid is encapsulated in a lipid.

53. The vaccine composition of any one of claims 46-50, further comprising an adjuvant.

54. A nucleic acid encoding an antigen of B. afzelii for use in preparation of a vaccine, wherein the nucleic acid has at least 90% identity to a nucleic acid sequence selected from the group consisting of SEQ ID NO:1, SEQ ID NO:3, and SEQ ID NO:5, or an antigenic fragment thereof.

55. The nucleic acid of claim 54 wherein the nucleic acid is an RNA transcript of the nucleic acid sequence selected from the group consisting of SEQ ID NO:1, SEQ ID NO:3, and SEQ ID NO: 5.


SEQUENCE LISTING (6 sequences)

SEQ ID NO: 1 - DNA, 1056 bp, Borrelia afzelii
  misc_feature 1..1056: note = D0016 Antigen
  source 1..1056: mol_type = genomic DNA
atgaaaattaaaagtaaatgcttagctttgggattgctttttggttttatcagttgtgatttattcataagagatgaaataaaagagaaatctcttggcttgtgtgatgaggaaagttctattttagagactggtgacaaatctgttaaaaagtctcttaataagaaaggcaaagataaggttgctagaaagaaagttgaaggtaatgctgttaaaaaagacccgtttaatcatcatgtaaagagggagtctgttaataatagtaatctatcacaaaaaaatgtgatatcggaagaagaaattttgaaaactaaattattaagagaacgacctgagactagaaaagaagaaatacaaaaacagcaagatgagcataaaaggatgcttcaaggaagtttaagttttcttagtggtgaaagtggtgaattgaaggatactattgaaagcaatgaaattgattttactatagattctgatttaagactgaagagtgatttacaagctatttcaggctcaaattctatttcatatactgatgaaatagaagaagaagattatgatcagtattctttagaagaagattattattatgatgaggaaacaagattaagtaatagatatgaatcttatctagagggtgttaaatataatgtaagttcagcaattaaaacaattgttaagatatatgataattataccttactttcaacaaagcaaacccaaatgtattctacacgtcttgacaaccttgctaaagccaaagctagagaagaagctaaaaagtttacaaaagaagaacttgaaaaagatcttaagaccttattgaactatattcaagtgagtgcaaggactgcgacaaattttgtatatgcaagagaaatatattcaaaaagaaaattagatgccattgaaacagaaataaaaaatttagttttaaagatcaaaggacaatctgatttatacgaggcatataaagcaatagtaaggtcaatcttattaatgaaagattctcttaaaataatcgaaatagtcattgataagaatggtgtttggtac

SEQ ID NO: 2 - protein, 352 aa, Borrelia afzelii
  REGION 1..352: note = MISC_FEATURE - D0016 Antigen
MKIKSKCLALGLLFGFISCDLFIRDEIKEKSLGLCDEESSILETGDKSVKKSLNKKGKDKVARKKVEGNAVKKDPFNHHVKRESVNNSNLSQKNVISEEEILKTKLLRERPETRKEEIQKQQDEHKRMLQGSLSFLSGESGELKDTIESNEIDFTIDSDLRLKSDLQAISGSNSISYTDEIEEEDYDQYSLEEDYYYDEETRLSNRYESYLEGVKYNVSSAIKTIVKIYDNYTLLSTKQTQMYSTRLDNLAKAKAREEAKKFTKEELEKDLKTLLNYIQVSARTATNFVYAREIYSKRKLDAIETEIKNLVLKIKGQSDLYEAYKAIVRSILLMKDSLKIIEIVIDKNGVWY

SEQ ID NO: 3 - DNA, 423 bp, Borrelia afzelii
  misc_feature 1..423: note = A0029 Antigen
  source 1..423: mol_type = genomic DNA
atgaaaaacagatttttttttatatatttatcatcagctttagtttttttgtttagctgtaatggttttttgacacttgaaagcaaagaaagatcaagctttacagataaacaaagaataagtattttagagaccatcaaaaaccactataatggcgactctgaaaaaatagctagagttgacaaatattttgatcgattagaggattctcaaaaagatgtttttttacaattgttttcggctggatttgactatataaactctaaagaatcagaacctaaagaatcagcaaatagaagagctaatttagatgctttaaaaagtaattttgaatccaaactaaaacagcttaattacacagaagaagattttaataaattggtagacgactttaatgagtttgttgattctaagtccacttct

SEQ ID NO: 4 - protein, 141 aa, Borrelia afzelii
  REGION 1..141: note = MISC_FEATURE - A0029 Antigen
MKNRFFFIYLSSALVFLFSCNGFLTLESKERSSFTDKQRISILETIKNHYNGDSEKIARVDKYFDRLEDSQKDVFLQLFSAGFDYINSKESEPKESANRRANLDALKSNFESKLKQLNYTEEDFNKLVDDFNEFVDSKSTS

SEQ ID NO: 5 - DNA, 552 bp, Borrelia afzelii
  misc_feature 1..552: note = A0001 Antigen
  source 1..552: mol_type = genomic DNA
atgaaaactttggttggcgcttgtatagtaacattggctgtattgggttgttatttgcctgataaacagaaacaagccgttcaaactttttttaagaattcacaaagtagtgatataggttctgatgagattgttgctgatgaaattgttgctgatgggatatttgataatttaaaattatatatgactgagcataatttgctagttgatataaaaaagaccataatcagtttaaaagatcctaattatcgtgctgtacccccagtgcgtgactataatgaggagtattttaataaattctttttagatttggggtctgagcgatctaaagaattgattaagttatttggcaagataaaaaatgagcagaatgatgataaatttaaaagagaagcttattggctatattcatgtataagggatttatattcttcagatattaagtattctggtgaaaacgggaatgagtatattgttgatatgcctaggaggcccactattgatcaacaatatcttaaagtgaagggagtaatagaacagtatgctttaaga

SEQ ID NO: 6 - protein, 184 aa, Borrelia afzelii
  REGION 1..184: note = MISC_FEATURE - A0001 Antigen
MKTLVGACIVTLAVLGCYLPDKQKQAVQTFFKNSQSSDIGSDEIVADEIVADGIFDNLKLYMTEHNLLVDIKKTIISLKDPNYRAVPPVRDYNEEYFNKFFLDLGSERSKELIKLFGKIKNEQNDDKFKREAYWLYSCIRDLYSSDIKYSGENGNEYIVDMPRRPTIDQQYLKVKGVIEQYALR